(19) United States
(12) Patent Application Publication
(10) Pub. No.: US 2024/0174420 A1
(43) Pub. Date: May 30, 2024

(54) FILM USED FOR OUTER BAG FOR CHEMICAL BODY WARMER

(71) Applicant: KOBAYASHI PHARMACEUTICAL CO., LTD., Osaka (JP)

(72) Inventor: Kentaro ASONUMA, Osaka-shi, Osaka (JP)

(21) Appl. No.: 18/553,444

(22) PCT Filed: Mar. 23, 2022
(86) PCT No.: PCT/JP2022/013659

§ 371 Date: Sep. 29, 2023

(30) Foreign Application Priority Data
Apr. 2, 2021 (JP) 2021-063528

(51) Int. Cl. B65D 65/40; A61F 7/02; A61F 7/03; B32B 27/08; B32B 27/32

(52) U.S. Cl. CPC B65D 65/40; A61F 7/034; B32B 27/08; B32B 27/32; A61F 2007/0222; A61F 2007/0258; B32B 2250/02; B32B 2250/242; B32B 2255/10; B32B 2255/20; B32B 2255/205; B32B 2307/308; B32B 2307/31; B32B 2307/518; B32B 2307/7244; B32B 2307/7246; B32B 2439/46

(57) ABSTRACT

This film for an outer bag is a film that is used in an outer bag for containing a chemical body warmer. The film contains at least a deposition film layer obtained by depositing a metal and/or a metal compound. and the film has an oxygen permeability. at 50° C. of 11.4 to 62.8 cc/m²⋅day⋅atm. The film forms the outer bag of a chemical body warmer.

### TECHNICAL FIELD

The present invention relates to a film used for an outer bag for accommodating a chemical body warmer, and a chemical body warmer in outer bag formed of the film and accommodating the chemical body warmer.

### BACKGROUND ART

A conventional chemical body warmer (so-called disposable body warmer) uses a heat generating mechanism in which an oxidizable metal such as an iron powder and the like generates oxidation heat by contact with oxygen, and has a structure in which a heat-generating composition containing an oxidizable metal is stored in a container (inner bag) having air permeability. In addition, since the chemical body warmer starts to generate heat when the oxidizable metal comes into contact with oxygen, the chemical body warmer is sealed by being accommodated in a container (outer bag) capable of blocking oxygen permeation until the start of use.

On the other hand, the chemical body warmer has a problem that when a high temperature condition is applied during storage, hydrogen gas is inevitably generated by the reaction of the oxidizable metal contained in the heat-generating composition, and the internal pressure in the outer bag increases to swell.

Therefore, conventionally, a technique of suppressing expansion of the outer bag due to generation of a hydrogen gas has been proposed focusing on the composition of the heat-generating composition of the chemical body warmer. For example, Patent Document 1 describes that the amount of a hydrogen gas generated can be reduced by including a reduced iron powder having a calcium content of 0.3 wt % or more in a heat-generating composition. In addition, Patent Document 2 describes that the generation amount of a hydrogen gas can be reduced by containing an iron powder for reactant in which the amount of wustite contained in the iron powder is 5% or less as an X-ray peak intensity ratio with iron in the heat-generating composition.

### PRIOR ART DOCUMENT

### Patent Documents

Patent Document 1: Japanese Patent Laid-open Publication No. 2008-222763

Patent Document 2: Japanese Patent Laid-open Publication No. H10-17907

### SUMMARY OF THE INVENTION

### Problems to be Solved by the Invention

Conventionally, in order to suppress expansion of an outer bag of a chemical body warmer during storage, the composition of a heat-generating composition has been improved, but studies focusing on the oxygen permeability of the outer bag have not been conducted. Therefore, the present inventor focused on the outer bag accommodating a chemical body warmer, and preliminarily verified the degree of expansion at the time of high-temperature storage, and found that the conventional outer bag may be expanded by high-temperature storage, and conversely, may be shrunk due to a decrease in the internal pressure of the outer bag.

Therefore, an object of the present invention is to provide an outer bag that is used for accommodating a chemical body warmer and can suppress swelling and shrinkage caused by a change in internal pressure such as generation of a hydrogen gas during storage, and the like.

A conventional outer bag for accommodating a chemical body warmer is required to have a gas barrier property, and in Japan, JIS 4100-1996 “Disposable body warmer” defines that an oxygen permeability at 23° C. is 12.7 cc/m²⋅day⋅atm or less as a standard. Therefore, conventionally, in the physical property evaluation of the material of an outer bag accommodating a chemical body warmer, attention has been paid to the oxygen permeability at 23° C., and studies focusing on the oxygen permeability in the high temperature range of higher than 23° C. have not been conducted.

Under such circumstances, the present inventors have extensively conducted studies for achieving the above-mentioned object, and resultantly found that when a film including a vapor-deposited film layer on which a metal and/or a metal compound is vapor-deposited and having an oxygen permeability of 11.4 to 62.8 cc/m²⋅day⋅atm at 50° C. is used as a material of an outer bag accommodating a chemical body warmer, swelling and shrinkage caused by a change in internal pressure such as generation of a hydrogen gas during storage and the like in the outer bag accommodating the chemical body warmer can be suppressed. The present invention has been completed by further conducting studies based on this finding.

That is, the present invention provides inventions of the following aspects.

Item 1. A film for outer bag used in an outer bag for accommodating a chemical body warmer,
wherein
the film contains at least a vapor-deposited film layer on which a metal and/or a metal compound is vapor-deposited, and
the film has an oxygen permeability at 50° C. of 11.4 to 62.8 cc/m²⋅day⋅atm.

Item 2. The film for outer bag according to item 1, wherein the film has an oxygen permeability at 40° C. of 5.7 to 26.7 cc/m²⋅day⋅atm.

Item 3. The film for outer bag according to item 1 or 2, wherein the film has a water vapor permeability at 40° C. of 5 g/m²⋅day or less.

Item 4. The film for outer bag according to any one of items 1 to 3, wherein the apor-deposited film layer is an aluminum vapor-deposited film or an alumina vapor-deposited film.

Item 5. The film for outer bag according to any one of items 1 to 4, wherein the vapor-deposited film layer is a polyolefin-based resin film on which a metal and/or a metal compound is vapor-deposited.

Item 6. The film for outer bag according to item 5, wherein the polyolefin-based resin film is a biaxially stretched polypropylene film.

Item 7. The film for outer bag according to any one of items 1 to 6, wherein a base material film layer, the vapor-deposited film layer, and a heat-weldable resin layer are laminated in this order.

Item 8. A chemical body warmer in outer bag, wherein the chemical body warmer is accommodated in an outer bag formed of the film for outer bag according to any one of items 1 to 7.

Item 9. Use of a film that contains at least a vapor-deposited film layer on which a metal and/or a metal compound is vapor-deposited and that has an oxygen permeability at 50° C. of 11.4 to 62.8 cc/m²⋅day⋅atm, as an outer bag for accommodating a chemical body warmer.

### Advantages of the Invention

According to the present invention, it is possible to effectively suppress swelling and shrinkage caused by a change in internal pressure such as generation of a hydrogen gas during storage, and the like, in an outer bag accommodating a chemical body warmer.

### BRIEF DESCRIPTION OF THE DRAWINGS

FIG. 1 is a schematic plan view of a sample (chemical body warmer accommodated in an outer bag) used for measurement of an expansion/shrinkage ratio.

FIG. 2 is a graph showing the results of obtaining a regression line by plotting the oxygen permeability at 50° C. on the vertical axis and the swelling/shrinkage ratio at 50° C. on the horizontal axis for Examples 1 to 3 and Comparative Examples 1 to 3.

FIG. 3 is a diagram showing the results of obtaining a regression line by plotting the oxygen permeability at 40° C. on the vertical axis and the swelling/shrinkage ratio at 40° C. on the horizontal axis for Examples 1 to 3 and Comparative Examples 1 to 3.

FIG. 4 is a graph showing a result of measuring a temporal change of a heat generation temperature after opening of a body warmer in outer bag stored at 40° C. for 3 months.

FIG. 5 is a graph showing a result of measuring a temporal change of a heat generation temperature after opening of a body warmer in outer bag stored at 50° C. for 3 months.

### EMBODIMENTS OF THE INVENTION

### 1. Film for Outer Bag

A film for outer bag of the present invention is a film used for forming an outer bag for accommodating a chemical body warmer, in which the film contains at least a vapor-deposited film layer on which a metal and/or a metal compound is vapor-deposited, and the film has an oxygen permeability at 50° C. of 11.4 to 62.8 cc/m²⋅day⋅atm. Hereinafter, the film for outer bag of the present invention will be described in detail.

### [Material and Layer Structure Used in Film for Outer Bag]

The film for outer bag of the present invention includes at least a vapor-deposited film layer on which a metal and/or a metal compound is vapor-deposited.

In the vapor-deposited film layer, the material for forming the vapor-deposited film may be a metal and/or a metal compound, and includes specifically metals such as aluminum, chromium, zinc, gold, silver, platinum, nickel, and the like; and metal compounds such as alumina, silica, titanium oxide, zirconium oxide, magnesium fluoride, and the like. These metals and metal compounds may be used singly or in combination of two or more kinds thereof.

Among these metals and metal compounds, aluminum and alumina are preferred, and aluminum is more preferred, from the viewpoint of suitably having a range of oxygen permeability at 40° C. and 50° C. described later and more effectively suppressing swelling and shrinkage during storage in an outer bag containing a chemical body warmer.

In the vapor-deposited film layer, the thickness of the vapor-deposited film of the metal and/or the metal compound is not particularly limited as long as it can satisfy the range of oxygen permeability at 40° C. and 50° C. described later, but it is, for example, 200 to 1000 Å, preferably 400 to 900 Å, and more preferably 500 to 800 Å.

In the vapor-deposited film layer, the material of the film supporting the vapor-deposited film is not particularly limited, and examples thereof include polyolefin-based resins such as polypropylene, polyethylene, ethylene-vinyl acetate copolymer, and the like: polyethylene terephthalate, polyacrylonitrile, ethylene-vinyl alcohol copolymer, polyamide, polyimide, polyurethane, polystyrene, polyvinyl alcohol, polyvinyl chloride, polyvinylidene chloride, polycarbonate, and the like. Among them, a polyolefin-based resin is preferred, and polypropylene is more preferred, from the viewpoint of suitably providing a range of oxygen permeability at 40° C. and 50° C. described later, and further effectively suppressing swelling and shrinkage at the time of storage in an outer bag accommodating a chemical body warmer.

In the vapor-deposited film layer, the film supporting the vapor-deposited film may be either a stretched film or an unstretched film, but is preferably a stretched film, and more preferably a biaxially stretched film.

In the vapor-deposited film layer, the thickness of the film that supports the vapor-deposited film is not particularly limited as long as it can satisfy the range of oxygen permeability at 40° C. and 50° C. described later, but it is, for example, 10 to 50 μm, preferably 15 to 40 μm, and more preferably 18 to 30 μm.

In the vapor-deposited film layer, a suitable example of the film that supports the vapor-deposited film is biaxially stretched polypropylene (OPP). By using biaxially stretched polypropylene on which a metal and/or a metal compound is vapor-deposited as the vapor-deposited film layer, it is possible to suitably have a range of oxygen permeability at 40° C. and 50° C. described later, and to remarkably suppress swelling and shrinkage during storage in an outer bag accommodating a chemical body warmer. Further, by using biaxially stretched polypropylene on which a metal and/or a metal compound is vapor-deposited, the water vapor permeability at 40° C. and 50° C. also becomes a low value, and excellent water vapor barrier properties can be provided even under high temperature conditions.

The vapor-deposited film layer can be formed by vapor-depositing a metal and/or a metal compound on a film to be a support by a known vapor deposition method such as a physical vapor deposition method, a chemical vapor deposition method, and the like.

The film for outer bag of the present invention preferably has a heat-weldable resin layer in addition to the vapor-deposited film layer. By including the heat-weldable resin layer, the peripheral edge portion can be thermally sealed and molded into a bag shape without using an adhesive. The heat-sealable resin layer is a layer formed of a heat-sealable resin, and is a layer disposed on one surface (inner surface in the case of an outer bag) of the film for outer bag. The heat-sealable resin layer may be laminated so as to be in contact with the vapor-deposited film of the vapor-deposited film layer, or may be laminated so as to be in contact with the film (support) of the vapor-deposited film layer.

The heat-sealable resin to be used for forming the heat-sealable resin layer is not particularly limited as long as it can be heat-sealed, and examples thereof include polyolefins such as polyethylene, polypropylene, norbornene, and the like, and carboxylic acid-modified polyolefins, and the like. These heat-sealable resins may be used singly or in combination of two or more kinds thereof. Among these heat-sealable resins, polyolefin is preferable, and polyethylene is more preferable. The thickness of the heat-sealable resin layer is not particularly limited as long as it can be heat-sealed, and is, for example, 10 to 150 μm, preferably 12 to 100 μm, and more preferably 15 to 90 μm.

In order to laminate the heat-sealable resin layer on the vapor-deposited film layer, a heat-sealable resin may be applied to the vapor-deposited film layer, or a film made of a heat-sealable resin may be bonded to the vapor-deposited film layer via an adhesive.

The film for outer bag of the present invention preferably has a base material film layer in addition to the vapor-deposited film layer. By including the base material film layer, strength and durability can be enhanced. The base material film layer is a layer formed of a resin film, and is a layer disposed on one surface (outside in the case of an outer bag) of the film for outer bag. The base material film layer may be laminated so as to be in contact with the vapor-deposited film of the vapor-deposited film layer, or may be laminated so as to be in contact with the film (support) of the vapor-deposited film layer.

The type of resin for forming the base material film layer is not particularly limited, and examples thereof include polypropylene, polyethylene, an ethylene-vinyl acetate copolymer, polyethylene terephthalate, polyacrylonitrile, an ethylene-vinyl alcohol copolymer, polyamide, polyimide, polyurethane, polystyrene, polyvinyl alcohol, polyvinyl chloride, polyvinylidene chloride, polycarbonate, and the like. Among them, polypropylene is preferable.

The film that forms the base material film layer may be either a stretched film or an unstretched film, but is preferably a stretched film, and more preferably a biaxially stretched film.

Preferable examples of the base material film layer include biaxially stretched polypropylene (OPP). When biaxially stretched polypropylene is used as the base material film layer, it is easy to adjust the oxygen permeability of the film for outer bag to a more appropriate range.

The thickness of the base material film layer is not particularly limited as long as it can satisfy the range of oxygen permeability at 40° C. and 50° C. described later, and is, for example, 10 to 50 μm, preferably 12 to 40 μm, and more preferably 15 to 30 μm.

In order to laminate the base material film layer on the vapor-deposited film layer, the vapor-deposited film layer and the base material film layer may be bonded to each other with an adhesive interposed therebetween.

One embodiment of the film for outer bag of the present invention includes a film not containing a high barrier layer other than the vapor-deposited film layer. Here, the high barrier layer is a layer that alone exhibits an oxygen permeability of 3 g/m²⋅day or less at 23° C., and corresponds to, for example, a metal foil layer, or the like. The oxygen permeability at 23° C. is a value measured by a method described later.

### [Oxygen Permeability of Film for Outer Bag]

The film for outer bag of the present invention satisfies an oxygen permeability of 11.4 to 62.8 cc/m²⋅day⋅atm at 50° C. By including the vapor-deposited film layer and satisfying such a range of oxygen permeability at 50° C., it is possible to suppress swelling and shrinkage due to a change in internal pressure during high temperature storage in an outer bag accommodating a chemical body warmer.

Without wishing a limited interpretation, it is considered that the film for outer bag of the present invention can suppress swelling and shrinkage caused by a change in internal pressure during high temperature storage based on the following mechanism. When the outer bag accommodating a chemical body warmer is exposed to a high temperature, a hydrogen gas or the like is generated from the chemical body warmer. When the oxygen permeability of the film for outer bag at a high temperature is low, the gas generated inside cannot be sufficiently released to the outside, the internal pressure of the outer bag increases and the outer bag expands, and when the internal pressure of the outer bag excessively increases, rupture of the outer bag may occur. On the other hand, when the oxygen permeability of the film for outer bag at a high temperature is high, a gas generated inside is rapidly released to the outside, the inside of the outer bag has a negative pressure, and the outer bag shrinks, leading to deterioration of appearance. In the film for outer bag of the present invention, when the oxygen permeability at 50° C. satisfies the above range, the generated gas can be released to the outside at an appropriate speed when the temperature becomes high during storage, and swelling and shrinkage due to a change in internal pressure can be suppressed.

The oxygen permeability at 50° C. of the film for outer bag of the present invention may be 11.4 to 62.8 cc/m²⋅day⋅atm, but is preferably 19.4 to 62.8 cc/m²⋅day⋅atm, more preferably 19.4 to 54.7 cc/m²⋅day⋅atm, and still more preferably 24.7 to 54.7 cc/m²⋅day⋅atm, from the standpoint of more effectively suppressing swelling and shrinkage during storage in an outer bag accommodating a chemical body warmer.

The oxygen permeability of the film for outer bag of the present invention at 40° C. is not particularly limited, and is, for example, 5.7 to 26.7 cc/m²⋅day⋅atm, preferably 5.7 to 23.6 cc/m²⋅day⋅atm, and more preferably 8.8 to 23.6 cc/m²⋅day⋅atm. When the oxygen permeability at 40° C. satisfies the above range, it is possible to more effectively suppress swelling and shrinkage during storage in an outer bag accommodating a chemical body warmer.

In the present invention, the oxygen permeability at 50° C. and 40° C. is a value obtained by using an oxygen gas at a relative humidity of 0% and measuring the amount of an oxygen gas permeated by a pressure sensor method in accordance with Japanese Industrial Standards JIS K7126-1: 2006 “Plastics—Film and sheeting—Determination of gas transmission rate—Part 2: Differential pressure method”.

The oxygen permeability at 23° C. of the film for outer bag of the present invention is not particularly limited as long as the oxygen permeability at 40° C. and 50° C. satisfies the above range, and examples thereof include 25.0 cc/m²⋅day⋅atm or less, preferably 15 cc/m²⋅day⋅atm or less, more preferably 12.7 cc/m²⋅day⋅atm or less, and still more preferably 1 to 8 cc/m²⋅day⋅atm.

In the present invention, the oxygen permeability at 23° C. is a value obtained by using an oxygen gas at a relative humidity of 60% and measuring the amount of the oxygen gas that permeates by an electrolytic sensor method in accordance with Japanese Industrial Standards JIS K7126-2: 2006 “Plastics—Film and sheeting—Determination of gas transmission rate—Part 2: Equal-pressure method”.

Since the oxygen permeability is greatly affected by the physical properties of the vapor-deposited film layer, the material and thickness of the vapor-deposited film in the vapor-deposited film layer to be used and the material and thickness of the film (support) in the vapor-deposited film layer may be appropriately adjusted in order to adjust the oxygen permeability at each temperature to be in the above range. In addition, since the base material film layer may also affect the oxygen permeability, the oxygen permeability at each temperature can be adjusted to fall within the above range by appropriately adjusting the material, thickness, and the like of the base material film layer to be used in addition to the physical properties of the vapor-deposited film layer.

### [Water Vapor Permeability of Film for Outer Bag]

In order to suppress release of water contained in the heat-generating composition of a chemical body warmer to the outside and to suppress entry of external water vapor into the outer bag, the film for outer bag of the present invention preferably has a low water vapor permeability and an excellent water vapor barrier property.

The water vapor permeability at 30° C. in the film for outer bag of the present invention is, for example, 10 g/m²⋅day or less, preferably 8 g/m²⋅day or less, more preferably 5 g/m²⋅day or less, still more preferably 3 g/m²⋅day or less, and particularly preferably 0.5 g/m²⋅day or less. The lower limit value of the water vapor permeability at 30° C. is not particularly limited, and is, for example, 0 g/m²⋅day or 0.1 g/m²⋅day.

In the film for outer bag of the present invention, the water vapor permeability at 40° C. is preferably 5 g/m²⋅day or less, more preferably 3.5 g/m²⋅day or less, and still more preferably 1 g/m²⋅day or less, from the standpoint of imparting an excellent water vapor barrier property at the time of storage at a high temperature. The lower limit value of the water vapor permeability at 40° C. is not particularly limited, and is, for example, 0 g/m²⋅day or 0.1 g/m²⋅day.

In the film for outer bag of the present invention, the water vapor permeability at 50° C. is preferably 5 g/m²⋅day or less, more preferably 4.5 g/m²⋅day or less, still more preferably 3 g/m²⋅day or less, and particularly preferably 1 g/m²⋅day or less from the viewpoint of imparting excellent water vapor barrier properties at the time of storage at a high temperature. The lower limit value of the water vapor permeability at 50° C. is not particularly limited, and is, for example, 0 g/m²⋅day or 0.1 g/m²⋅day.

In the present invention, the water vapor permeability at 30° C., 40° C., and 50° C. is a value measured under a condition of a relative humidity of 90% in accordance with Japanese Industrial Standard JIS Z0208-1976 “Testing methods for determination of the water vapor transmission rate of moisture-proof packaging materials (dish method)”.

Since the water vapor permeability is greatly affected by the physical properties and structure of the vapor-deposited film layer, the material and thickness of the vapor-deposited film in the vapor-deposited film layer to be used and the material and thickness of the film (support) in the vapor-deposited film layer may be appropriately adjusted in order to adjust the water vapor permeability at each temperature to fall within the above range. In addition, since the base material film layer may also affect the water vapor permeability, the water vapor permeability at each temperature can be adjusted to fall within the above range by appropriately adjusting the material, thickness, and the like of the base material film layer to be used in addition to the physical properties of the vapor-deposited film layer.

### 2. Outer Bag for Chemical Body Warmer

The film for outer bag of the present invention is used for an outer bag for accommodating a chemical body warmer. That is, the film for outer bag of the present invention may be formed into a bag shape to hermetically seal a chemical body warmer to obtain a chemical body warmer in outer bag.

In order to make the film for outer bag of the present invention into a bag shape, the peripheral edge portion may be bonded in a state where two films for outer bag having a predetermined shape are overlapped, or the peripheral edge portion may be bonded in a state where one film for outer bag having a predetermined shape is bent. When the film for outer bag of the present invention is provided with a heat sealing layer, the film for outer bag can be bonded by heat sealing of the heat sealing layer, and when the film for outer bag of the present invention is not provided with a heat sealing layer, the film for outer bag can be bonded by using an adhesive.

The type of the chemical body warmer accommodated in the outer bag formed of the film for outer bag of the present invention is not particularly limited, and examples thereof include a chemical body warmer in which a heat-generating composition containing an oxidizable metal is accommodated in a container (inner bag) having air permeability.

The type of the oxidizable metal contained in the heat-generating composition is not particularly limited as long as it can generate heat by oxidation, and examples thereof include metals such as iron (reduced iron, cast iron, atomized iron, electrolytic iron), aluminum, zinc, manganese, magnesium, calcium, and the like. These oxidizable metals may be used singly or in combination of two or more kinds thereof. The shape of the oxidizable metal is not particularly limited, but it is preferably in the form of powder, granule, or fiber, and more preferably in the form of powder from the viewpoint of heat generation efficiency. The content of the oxidizable metal in the heat-generating composition is appropriately set according to the heat generating property to be imparted, and is, for example, 20 to 80 wt %, preferably 25 to 70 wt %, and more preferably 45 to 60 wt %.

The heat-generating composition may contain an oxidation accelerator as necessary. The oxidation accelerator plays a role of retaining oxygen and supplying oxygen to the oxidizable metal. The type of the oxidation accelerator is not particularly limited as long as oxygen can be retained and oxygen can be supplied to the oxidizable metal, and examples thereof include carbon materials such as activated carbon, carbon black, acetylene black, bamboo charcoal, charcoal, coffee grounds charcoal, graphite, coal, coconut husk charcoal, almond green charcoal, peat, lignite coal, and the like. These oxidation accelerators may be used singly or in combination of two or more kinds thereof. Among these oxidation accelerators, activated carbon, carbon black, bamboo charcoal, charcoal, and coffee grounds charcoal are preferred, and activated carbon is further preferred. The shape of the oxidation accelerator is not particularly limited, but it is preferably in the form of powder, granule, or fiber, and more preferably in the form of powder from the viewpoint of heat generation efficiency. The content of the oxidation accelerator in the heat-generating composition is appropriately set according to the heat generating property to be imparted, and the like, and is, for example, 1 to 30 wt %, preferably 3 to 25 wt %, and further preferably 4 to 25 wt %.

Further, the heat-generating composition may contain water as necessary. Water plays a role of oxidizing the oxidizable metal together with oxygen. As water, any of distilled water, ion-exchanged water, pure water, ultrapure water, tap water, industrial water, and the like may be used. The content of water in the heat-generating composition is appropriately set according to the heat generating property to be imparted, and is, for example, 5 to 50 wt %, preferably 10 to 40 wt %, and further preferably 15 to 35 wt %.

The heat-generating composition may further contain water-soluble salts as necessary. When a water-soluble salt is contained, oxidation of the oxidizable metal can be promoted. The kind of the water-soluble salts is not particularly limited, and examples thereof include sulfates, hydrogencarbonates, chlorides, hydroxides, or the like of alkali metals (sodium, potassium, etc.), alkaline earth metals (calcium, magnesium, etc.) and heavy metals (iron, copper, aluminum, zinc, nickel, silver, barium, and the like). Among these water-soluble salts, chlorides such as sodium chloride, potassium chloride, calcium chloride, magnesium chloride, iron (first and second) chloride, and the like are preferable, and sodium chloride and the like are more preferable from the viewpoint of conductivity, chemical stability, and the like. These water-soluble salts may be used singly or in combination of two or more kinds thereof. The content of the water-soluble salt in the heat-generating composition is appropriately set according to the heat generating property to be imparted, and is, for example, 0.1 to 10 wt %, preferably 0.5 to 7 wt %, and further preferably 1 to 5 wt %.

The heat-generating composition may contain a humectant as necessary. The humectant plays a role of retaining water and supplying water to the oxidation reaction field. The kind of the humectant is not particularly limited, and examples thereof include inorganic porous substances such as vermiculite (vermiculite), perlite, calcium silicate, magnesium silicate, kaolin, talc, smectite, mica, bentonite, calcium carbonate, silica gel, alumina, zeolite, silicon dioxide, diatomaceous earth, and the like: organic substances such as pulp, wood flour (sawdust), cotton, starches, celluloses, and the like: water-absorbent resins such as a polyacrylic acid-based resin, a polysulfonic acid-based resin, a maleic anhydride-based resin, a polyacrylamide-based resin, a polyvinyl alcohol-based resin, a polyethylene oxide-based resin, a polyaspartic acid-based resin, a polyglutamic acid-based resin, a polyalginic acid-based resin, and the like; etc. These humectants may be used singly or in combination of two or more kinds thereof. Among these humectants, vermiculite, a polyacrylic acid-based resin, wood flour, and pulp are preferable; and more preferable examples thereof include vermiculite and a polyacrylic acid-based resin. The content of the humectant in the heat-generating composition is appropriately set according to the heat generating property to be imparted, and is, for example, 1 to 20 wt %, preferably 3 to 15 wt %, and further preferably 5 to 10 wt %.

If necessary, the heat-generating composition may further contain other additives such as a sequestering agent, a perfume, a thickener, an excipient, a surfactant, a hydrogen generation inhibitor, and the like.

The heat-generating composition can be prepared by mixing a predetermined amount of the above-described components. The heat-generating composition may be prepared in the presence of oxygen, but is preferably prepared under reduced pressure or under an inert gas atmosphere.

In the chemical body warmer, the container (inner bag) for accommodating the heat-generating composition only needs to have air permeability in at least a part thereof, and those conventionally used as a container of a chemical body warmer can be used.

As a preferred example of the container, there is a container in which a peripheral portion of an air-permeable first sheet and a peripheral portion of an air-permeable or air-impermeable second sheet are bonded to each other. Specific examples of the first sheet having air permeability include a nonwoven fabric, a woven fabric, a resin sheet having air permeability, a laminated sheet in which a nonwoven fabric or a woven fabric and a resin sheet having air permeability are laminated, and the like. When the second sheet has air permeability, the structure, material, water vapor permeability, and the like of the second sheet are the same as those of the first sheet. When the second sheet is air-impermeable, it may be specifically a sheet having at least one air-impermeable layer, and examples thereof include an air-impermeable resin sheet, a laminated sheet in which a nonwoven fabric or a woven fabric and an air-impermeable resin sheet are laminated, and the like.

The chemical body warmer may be provided with an adhesive layer on one surface of the container. When the adhesive layer is provided on a chemical body warmer, the surface of the adhesive layer may be covered with a peelable release sheet and accommodated in an outer bag.

### EXAMPLES

Hereinafter, the present invention will be described more specifically with reference to examples and the like, but the present invention is not limited thereto.

Hereinafter, the biaxially stretched polypropylene film may be abbreviated as “OPP”, the polyethylene as “PE”, and the polyethylene terephthalate film as “PET”.

### 1. Preparation of Film for Outer Bag

### Example 1

A film for outer bag was prepared in which an OPP layer (thickness: 19 μm), an aluminum vapor-deposited OPP layer (thickness: 19 μm), and a PE layer (thickness: 80 μm) were laminated in this order. In the film for outer bag, the aluminum vapor-deposited film of the aluminum vapor-deposited OPP layer is disposed so as to be in contact with the OPP layer, and the OPP layer and the aluminum vapor-deposited OPP layer are bonded to each other, and the aluminum vapor-deposited OPP layer and the PE layer are bonded to each other by a dry lamination method.

### Example 2

A film for outer bag was prepared in which an OPP layer (thickness: 19 μm), an aluminum vapor-deposited OPP layer (thickness: 19 μm), and a PE layer (thickness: 25 μm) were laminated in this order. In the film for outer bag, the aluminum vapor-deposited film of the aluminum vapor-deposited OPP layer is disposed so as to be in contact with the OPP layer, and the OPP layer and the aluminum vapor-deposited OPP layer are bonded to each other, and the aluminum vapor-deposited OPP layer and the PE layer are bonded to each other by a dry lamination method. The aluminum vapor-deposited OPP layer used in Example 2 is different from the aluminum vapor-deposited OPP layer used in Example 1.

### Example 3

A film for outer bag was prepared in which an alumina vapor-deposited OPP layer (thickness: 20 μm) and a PE layer (thickness: 45 μm) were laminated in this order. In the film for outer bag, the alumina vapor-deposited film of the alumina vapor-deposited OPP layer is disposed so as to be in contact with the OPP layer, and the alumina vapor-deposited OPP layer and the PE layer are bonded to each other by a dry lamination method.

### Comparative Example 1

A film for outer bag was prepared in which an OPP layer (thickness: 20 μm), a PE layer (thickness 15 μm; hereinafter, “first PE layer”), an alumina vapor-deposited PET layer (thickness: 12 μm), and a PE layer (thickness 20 μm; hereinafter, “second PE layer”) were laminated in this order. In the film for outer bag, the alumina vapor-deposited film of the alumina vapor-deposited PET layer is disposed so as to be in contact with the first PE layer. The OPP layer, the first PE layer, and the alumina vapor-deposited PET layer are bonded by performing a sandwich lamination method while extruding PE for forming the first PE layer. The alumina vapor-deposited PET layer and the second PE layer are bonded to each other by a single lamination method.

### Comparative Example 2

A film for outer bag was prepared in which an OPP layer (thickness: 20 μm), an alumina vapor-deposited PET layer (thickness: 12 μm), and a PE layer (thickness: 15 μm) were laminated in this order. In the film for outer bag, the alumina vapor-deposited film of the alumina vapor-deposited PET layer is disposed so as to be in contact with the OPP layer, and adhesion between the OPP layer and the alumina vapor-deposited PET layer and adhesion between the alumina vapor-deposited PET layer and the PE layer are achieved by a dry lamination method. The alumina vapor-deposited PET layer used in Comparative Example 2 is different from the alumina vapor-deposited PET layer used in Comparative Example 1.

### Comparative Example 3

A film for outer bag was prepared in which an OPP layer (thickness: 19 μm), an aluminum vapor-deposited PET layer (thickness: 12 μm), and a PE layer (thickness: 35 μm) were laminated in this order. In the film for outer bag, the aluminum vapor-deposited film of the aluminum vapor-deposited PET layer is disposed so as to be in layer contact with the OPP layer, and the OPP layer and the aluminum vapor-deposited PET layer are bonded to each other, and the aluminum vapor-deposited PET layer and the PE layer are bonded to each other by a dry lamination method.

### Comparative Example 4

A film for outer bag was prepared in which an OPP layer coated with polyvinylidene chloride (thickness: 30 μm), a PE layer (thickness 15 μm; hereinafter, “first PE layer”), and a PE layer (thickness 15 μm; hereinafter, “second PE layer”) were laminated in this order. In the film for outer bag, the polyvinylidene chloride coating film side of the OPP layer coated with polyvinylidene chloride is disposed so as to be in contact with the first PE layer. The OPP layer coated with polyvinylidene chloride, the first PE layer, and the second PE layer are bonded by performing a sandwich lamination method while extruding PE for forming the first PE layer.

### 2. Preparation of Chemical Body Warmer

A heat-generating composition having the composition shown in Table 1 was prepared. A chemical body warmer was produced by housing 21.2 g of the obtained heat-generating composition in a container formed of a laminated sheet in which a nylon spunbond (basis weight: 35 g/m²), a polyethylene layer (thickness: 20 μm), and a metallocene polyethylene layer (thickness: 20 μm) were laminated in this order. The container is a rectangular bag having a size of 50 mm×90 mm in a plan view, and an end of the container is heat-sealed so that the contained heat-generating composition does not leak.

TABLE 1

| | Blending compound |
|---|---|
| Blended component | (wt %) |
| Iron powder | 55 |
| Vermiculite | 4 |
| Water-absorbent resin | 2 |
| Water-soluble salt | 1.5 |
| Water | 24 |
| Activated carbon | 13.5 |
| Total | 100 |

### 3. Preparation of Chemical Body Warmer in Outer Bag

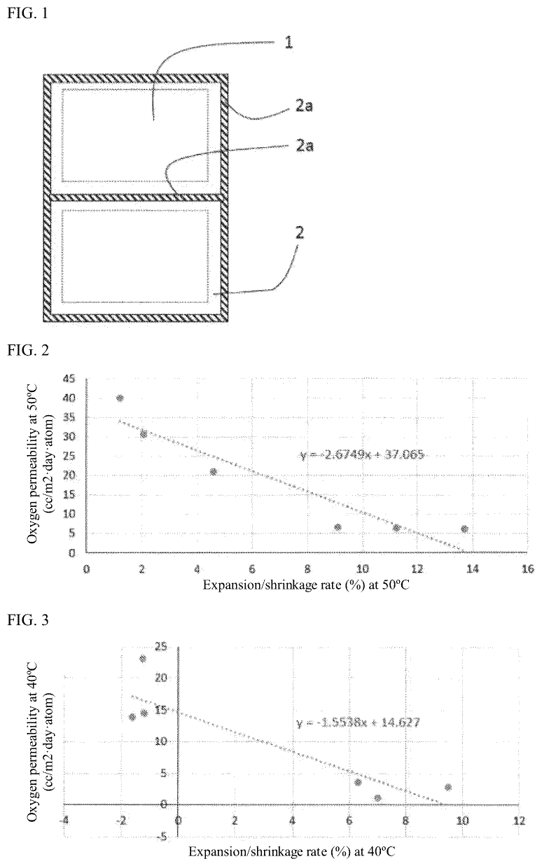

Two films for outer bag were prepared by cutting each of the film for outer bags into a rectangle of 120 mm×160 mm. On one film for outer bag, the two chemical body warmers were evenly arranged in the long side direction of the film for outer bag, and another film for outer bag was put thereon. At this time, the two films for outer bag were disposed so that the PE layers (heat-sealable resin layers) faced each other, and the OPP layer (base material resin layer) was disposed outside the outer bag. In this state, the end of the film for outer bag and the center of the film for outer bag in the long side direction were heat-sealed in the short side direction, whereby the chemical body warmer was accommodated in the outer bag and sealed. FIG. 1 shows a schematic plan view of a chemical body warmer (chemical body warmer in outer bag) accommodated in an outer bag. The operation of accommodating a chemical body warmer in an outer bag was quickly performed immediately after the production of the chemical body warmer.

### 4. Method for Measuring Oxygen Permeability of Film for Outer Bag

Oxygen permeabilities of the films for outer bag of Examples 1 to 3 and Comparative Examples 1 to 3 were measured under temperature conditions of 23° C., 40° C., and 50° C.

The oxygen permeability at 23° C. was measured by using an oxygen gas at a relative humidity of 60% and measuring the amount of an oxygen gas permeated by an electrolytic sensor method in accordance with Japanese Industrial Standard JIS K7126-2: 2006 “Plastics—Film and sheeting—Determination of gas transmission rate—Part 2: Equal-pressure method”. The measurement was performed by placing the back side of the film for outer bag (the outermost layer side made of PE) on the chamber side into which an oxygen gas was introduced.

The oxygen permeability at 40° C. and 50° C. was measured by using an oxygen gas at a relative humidity of 0% and measuring the amount of an oxygen gas permeated by a pressure sensor method in accordance with Japanese Industrial Standards JIS K7126-1: 2006 “Plastics—Film and sheeting—Determination of gas transmission rate—Part 2: Differential pressure method”. The measurement was performed by placing the back side of the film for outer bag (the outermost layer side made of PE) on the chamber side into which an oxygen gas was introduced.

### 5. Method for Measuring Water Vapor Permeability of Film for Outer Bag

The water vapor permeabilities of the films for outer bag of Examples 1 to 3 and Comparative Examples 1 to 4 were measured under temperature conditions of 30° C., 40° C., and 50° C. The water vapor permeability was measured in accordance with Japanese Industrial Standard JIS Z0208-1976 “Testing methods for determination of the water vapor transmission rate of moisture-proof packaging materials (dish method)”, under the condition of a relative humidity of 90%, in such a manner that the heat-sealing resin layer side of the film for outer bag was located inside the cup.

### 6. Measurement of Expansion/Shrinkage Rate of Outer Bag Accommodating Chemical Body Warmer

The chemical body warmer in outer bag using the film for outer bag of Examples 1 to 3 and Comparative Examples 1 to 3 was stored in an atmosphere of 40° C. and a relative humidity of 75% and an atmosphere of 50° C. and a relative humidity of 50% for 6 weeks. The degree of puffing of the outer bag before storage and after storage was measured by the following method.

A bucket capable of sufficiently accommodating a chemical body warmer in outer bag was prepared. Water was added into the bucket in an amount that the outer bag accommodating the chemical body warmer could be sufficiently immersed and that did not spill from the bucket even when the outer bag was immersed. The weight (weight A) in this state was measured with an electronic balance.

Next, the end of the chemical body warmer in outer bag was sandwiched between two magnets and immersed in the bucket, and the weight (weight B: weight of bucket containing water, chemical body warmer in outer bag, and two magnets) in this state was measured with an electronic balance. Furthermore, the weight (weight C) of the two magnets was measured with an electronic balance. By subtracting the weight A and the weight C from the weight B, the volume value in the outer bag accommodating the chemical body warmer was determined. Thus, the volume values before storage and 6 weeks after storage were measured, and the expansion/shrinkage ratio (%) was calculated according to the following formula. This measurement is a volumetric method using the Archimedes' principle, and when the swelling/shrinkage ratio is a positive value, it indicates that the outer bag swells at the end of the test as compared with the time of starting the test, and when the swelling/shrinkage ratio is a negative value, it indicates that the outer bag shrinks at the end of the test as compared with the time of starting the test.

$$[ Mathematical formula 1 ]$$

$$Expansion / shrinkage rate ( \% ) = \{ ( volume value after 6 weeks storage -$$

$$volume value before storage ) / ( volume value before storage ) \} \times 100$$

### 7. Measurement of Expansion/Shrinkage Rate of Outer Bag Accommodating Chemical Body Warmer

The storage stability of the chemical body warmer in outer bag using the film for outer bag of Example 2 and Comparative Example 3 was evaluated. Specifically, the chemical body warmer in outer bag was stored at 40° C. or 50° C. for 3 months, then the chemical body warmer was taken out from the outer bag, and the heat generating property was measured according to the “non-wearing” measurement method described in “6.6 Temperature characteristic” of Japanese Industrial Standards JIS S 4100-1996, “Disposable body warmers”. For comparison, the heat generating property of a chemical body warmer (control) immediately after the preparation was measured by the same method. This test was performed with n=6.

### 8. Results

Table 2 shows the results of measuring the oxygen permeability and the water vapor permeability (23° C.) of the film for outer bags of Examples 1 to 3 and Comparative Examples 1 to 3, and the expansion/shrinkage rate (%) of the chemical body warmer in outer bag formed of these films for outer bag. When the film for outer bag of Comparative Examples 1 to 3 was used, the swelling/shrinkage ratio at 50° C. was more than +9%, and the swelling/shrinkage ratio at 40° C. was also more than +5%, and thus puffing could not be sufficiently suppressed. On the other hand, in the case of using the film for outer bag of Examples 1 to 3, the swelling/shrinkage ratio at 50° C. was within the range of −10 to +9%, and the swelling/shrinkage ratio at 40° C. was within the range of +5%, and the effect of suppressing puffing was excellent.

For Examples 1 to 3 and Comparative Examples 1 to 3, a regression line was obtained by plotting the oxygen permeability at 50° C. on the vertical axis and the swelling/shrinkage rate at 50° C. on the horizontal axis, and the results are shown in FIG. 2. For Examples 1 to 3 and Comparative Examples 1 to 3, a regression line was obtained by plotting the oxygen permeability at 40° C. on the vertical axis and the swelling/shrinkage rate at 40° C. on the horizontal axis, and the results are shown in FIG. 3. As can be seen from FIGS. 2 and 3, there is a correlation between the oxygen permeability and the swelling/shrinkage ratio, and it has been found that the swelling/shrinkage ratio at 50° C. and 40° C. can be estimated by the following Formulas (1) and (2).

$$[ Mathematical formula 2 ]$$

$${y}_{1} = - 2.6749 {x}_{1} + 47.065 \qquad ( 1 )$$

$${y}_{2} = - 1.5538 {x}_{2} + 14.627 \qquad ( 2 )$$

$${y}_{1} : value of oxygen permeability ( cc / {m}^{2} \cdot day \cdot atom ) at 50 ° C . \\ {x}_{1} : expansion / shrinkage rate ( \% ) at 50 ° C . standard error of {y}_{1} : 5.59 \\ {y}_{2} : value of oxygen permeability ( cc / {m}^{2} \cdot day \cdot atom ) at 40 ° C . \\ {x}_{2} : expansion / shrinkage rate ( \% ) at 40 ° C . standard error of {y}_{2} : 4.3$$

On the basis of the above formula (1) and the standard error of y₁ (the value of the oxygen permeability at 50° C.), it has become clear that, in order to set the swelling/shrinkage ratio (%) at 50° C. within a range of about ±8%, the outer bag may be formed of a film for outer bag having an oxygen permeability of 11.4 to 62.8 cc/m²⋅day⋅atm at 50° C., in order to set the swelling/shrinkage ratio (%) at 50° C. within a range of about +5% to about −8%, the outer bag may be formed of a film for outer bag having an oxygen permeability of 19.4 to 62.8 cc/m²⋅day⋅atm at 50° C., in order to set the swelling/shrinkage ratio (%) at 50° C. within a range of about ±5%, the outer bag may be formed of a film for outer bag having an oxygen permeability of 19.4 to 54.7 cc/m²⋅day⋅atm at 50° C., and in order to set the swelling/shrinkage ratio (%) at 50° C. within the range of about +3% to about −5%, the outer bag may be formed of a film for outer bag having an oxygen permeability of 24.7 to 54.7 cc/m²⋅day⋅atm at 50° C.

On the basis of the above formula (2) and the standard error of y₂ (the value of the oxygen permeability at 40° C.), it has become clear that, in order to set the swelling/shrinkage ratio (%) at 40° C. within the range of about +3% to about −5%, the outer bag may be formed of a film for outer bag having an oxygen permeability of 5.7 to 26.7 cc/m²⋅day⋅atm at 40° C., in order to set the swelling/shrinkage ratio (%) at 40° C. within the range of about ±3%, the outer bag may be formed of a film for outer bag having an oxygen permeability of 5.7 to 23.6 cc/m²⋅day⋅atm at 40° C., and in order to set the swelling/shrinkage ratio (%) at 40° C. within the range of about +1% to about −3%, the outer bag may be formed of a film for outer bag having an oxygen permeability of 8.8 to 23.6 cc/m²⋅day⋅atm at 40° C.

TABLE 2

| | | Water vapor | | | | | |
|---|---|---|---|---|---|---|---|
| Film for | Vapor-deposited film contained | permeability | Oxygen permeability (cc/m² · day · atom) | | | Expansion/shrinkage rate (%) | |
| outer bag | in film for outer bag | (40° C.) (g/m² · day) | 23° C. | 40° C. | 50° C. | 40° C. | 50° C. |
| Example 1 | Aluminum vapor-deposited OPP | 0.3 | 6.8 | 23.0 | 40.0 | −1.26 | +1.21 |
| Example 2 | Aluminum vapor-deposited OPP | 0.2 | 6.8 | 14.5 | 30.6 | −1.20 | +2.07 |
| Example 3 | Alumina vapor-deposited OPP | 3.1 | 5.04 | 13.8 | 20.8 | −1.60 | +4.59 |
| Comparative | Alumina vapor-deposited PET | 1.0 | 1.5 | 3.5 | 6.5 | +6.32 | +9.12 |
| Example 1 | | | | | | | |
| Comparative | Alumina vapor-deposited PET | 1.3 | 1.0 | 1.0 | 6.2 | +7.00 | +11.26 |
| Example 2 | | | | | | | |
| Comparative | Aluminum vapor-deposited PET | 5.0 | 1.0 | 2.8 | 6.0 | +9.49 | +13.71 |
| Example 3 | | | | | | | |

Table 3 shows the results of measuring the oxygen permeability at 30°0 C., 40° C., and 50° C. for the films for outer bag of Examples 1 to 3 and Comparative Examples 1 to 4. As a result, it became clear that, in the films for outer bag of Examples 1 to 3 containing an aluminum vapor-deposited OPP, a low value of oxygen permeability could be maintained within a range of 30 to 50° C., and an excellent gas barrier property could be exhibited even under a high temperature by containing an aluminum vapor-deposited OPP.

TABLE 3

| Film for | Vapor-deposited film contained | Water vapor permeability (g/m² • day) | | |
|---|---|---|---|---|
| outer bag | in film for outer bag | 30° C. | 40° C. | 50° C. |
| Example 1 | Aluminum vapor-deposited OPP | 0.4 | 0.5 | 0.6 |
| Example 2 | Aluminum vapor-deposited OPP | 0.4 | 0.5 | 0.6 |
| Example 3 | Alumina vapor-deposited OPP | 1.1 | 3.3 | 4.3 |
| Comparative | Alumina vapor-deposited PET | 1.7 | 2.4 | 2.7 |
| Example 1 | | | | |
| Comparative | Alumina vapor-deposited PET | 1.1 | 2.4 | 4.3 |
| Example 2 | | | | |
| Comparative | Aluminum vapor-deposited PET | 0.4 | 1.1 | 1.7 |
| Example 3 | | | | |
| Comparative | - (polyvinylidene chloride - | 0.9 | 4.7 | 7.8 |
| Example 4 | coated OPP) | | | |

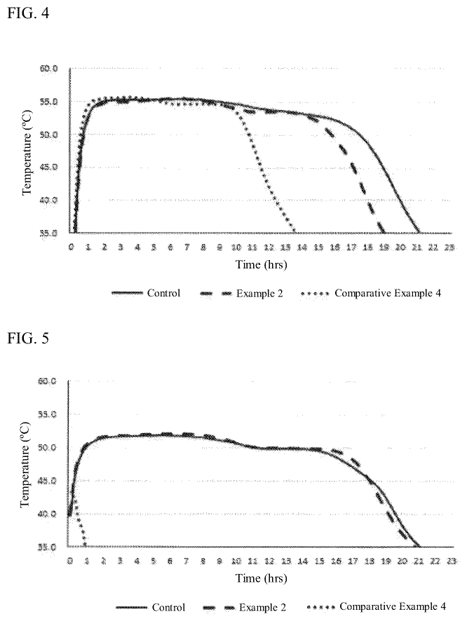

FIGS. 4 and 5 and Table 4 show the results of evaluating the heat generation characteristic of the chemical body warmer in outer bag formed of the film for outer bag of Example 2 and Comparative Example 4. Specifically, FIG. 4 shows a result of measuring a temporal change in the exothermic temperature after opening of a body warmer in outer bag stored at 40° C. for 3 months; FIG. 5 shows a result of measuring a temporal change in the exothermic temperature after opening of a body warmer in outer bag stored at 50° C. for 3 months; Table 4 shows the maximum temperature, duration, and average temperature after opening of a body warmer in outer bag stored at 40° C. for 3 months; and Table 5 shows the maximum temperature, duration, and average temperature after opening of a body warmer in outer bag stored at 50° C. for 3 months. The “maximum temperature” is the highest temperature while the chemical body warmer is generating heat, the “duration” is the time from higher than 40° C. to lower than 40° C., and the “average temperature” is the average temperature during the duration.

As a result, in the chemical body warmer in outer bag formed of the film for outer bag of Comparative Example 4, when stored at 40° C. for 3 months, the duration decreased as compared with the control (immediately after preparation), and when stored at 50° C. for 3 months, the maximum temperature, the duration, and the average temperature significantly decreased as compared with the control. On the other hand, in the chemical body warmer in outer bag formed of the film for outer bag of Example 2, even when stored at 40° C. and 50° C. for 3 months, the heat generating property equivalent to those of the control could be maintained.

TABLE 4

| | Stored for 3 months at 40° C. | | |
|---|---|---|---|
| | Maximum | | |
| | temperature | | Average |
| | (° C.) | Duration (hrs) | temperature (° C.) |
| Control (immediately | 55.4 | 20.0 | 53.0 |
| after preparation) | | | |
| Example 2 | 55.5 | 18.0 | 53.0 |
| Comparative Example 4 | 55.6 | 12.0 | 53.0 |

TABLE 5

| | Stored for 3 months at 50° C. | | |
|---|---|---|---|
| | Maximum | | Average |
| | temperature | | |
| | (° C.) | Duration (hrs) | temperature (° C.) |
| Control (immediately | 51.8 | 19.2 | 49.8 |
| after preparation) | | | |
| Example 2 | 52.1 | 19.1 | 49.9 |
| Comparative Example 4 | 45.2 | 0.4 | 41.7 |

### DESCRIPTION OF REFERENCE SIGNS

1: Chemical body warmer

2: Outer bag

2a: Heat seal portion

1. A film for outer bag used in an outer bag for accommodating a chemical body warmer, wherein the film contains at least a vapor-deposited film layer on which a metal and/or a metal compound is vapor-deposited, and the film has an oxygen permeability at 50° C. of 11.4 to 62.8 cc/m²⋅day⋅atm.

2. The film for outer bag according to claim 1, wherein the film has an oxygen permeability at 40° C. of 5.7 to 26.7 cc/m²⋅day⋅atm.

3. The film for outer bag according to claim 1, wherein the film has a water vapor permeability at 40° C. of 5 g/m²⋅day or less.

4. The film for outer bag according to claim 1, wherein the vapor-deposited film layer is an aluminum vapor-deposited film or an alumina vapor-deposited film.

5. The film for outer bag according to claim 1, wherein the vapor-deposited film layer is a polyolefin-based resin film on which a metal and/or a metal compound is vapor-deposited.

6. The film for outer bag according to claim 5, wherein the polyolefin-based resin film is a biaxially stretched polypropylene film.

7. The film for outer bag according to claim 1, wherein a base material film layer, the vapor-deposited film layer, and a heat-weldable resin layer are laminated in this order.

8. A chemical body warmer in outer bag, wherein the chemical body warmer is accommodated in an outer bag formed of the film for outer bag according to claim 1.
